(19) United States
(12) Patent Application Publication
(10) Pub. No.: US 2009/0177191 A1
(43) Pub. Date: Jul. 9, 2009

(54) Laser surgery methods and apparatus

(76) Inventor: Joe D. Brown, Panama City Beach, FL (US)

Correspondence Address: BACON & THOMAS, PLLC, 625 SLATERS LANE, FOURTH FLOOR, ALEXANDRIA, VA, 22314-1176, US

(21) Appl. No.: 12/314,517

(22) Filed: Dec. 11, 2008

Related U.S. Application Data
(63) Continuation-in-part of application No. 12/073,922, filed on Mar. 12, 2008.
(60) Provisional application No. 60/996,919, filed on Dec. 11, 2007.
(60) Provisional application No. 61/006,077, filed on Dec. 18, 2007.
(60) Provisional application No. 61/006,529, filed on Jan. 18, 2008.
(60) Provisional application No. 61/006,664, filed on Jan. 25, 2008.
(60) Provisional application No. 60/996,918, filed on Dec. 11, 2007.

(51) Int. Cl. A61B 18/24; A61B 18/22

(52) U.S. Cl. 606/12; 606/15

(57) ABSTRACT

A temperature of a laser treatment area is monitored and an output of a laser is controlled or modulated to prevent damage resulting from thermal runaway or burn back. The detector may be positioned proximate or at a distal end of the fiber, and may be in the form of a detector arranged to detect leakage of higher order propagation modes from a bend in the fiber. Manual or automatic cleaning of the fiber may be initiated upon detection of overheating, either by dipping the fiber in a cleaning solution or supplying cleaning fluid to the fiber or treatment area. A fiber position detector may also be included to prevent damage when the fiber is withdrawn into an introducer or catheter. UV radiation may be applied to the treatment site to prevent closing down of blood vessels upon introduction of a catheter or the bare fiber.

This application is a continuation-in-part of U.S. patent application Ser. No. 12/073,922, filed Mar. 12, 2008, which claims the benefit of U.S. Provisional Application Ser. Nos. 60/996,919, filed Dec. 11, 2007; 61/006,077, filed Jan. 18, 2008; 61/006,529, filed Jan. 18, 2008; and 61/006,664, filed Jan. 25, 2008. In addition, this application claims the benefit of U.S. Provisional Application Ser. No. 60/996,918, filed Dec. 11, 2007.

### BACKGROUND OF THE INVENTION

1. Field of the Invention

This invention relates to methods and apparatus for delivering energy to a tissue, and in particular to methods and apparatus for minimizing damage caused by overheating or thermal runaway at a treatment site.

More specifically, the invention relates to various improvements to the radiation feedback system disclosed in U.S. Pat. No. 5,098,427 (hereinafter the Dornier patent). The improvements include, but are not limited to, the following modifications of the Dornier system:
detection of radiation emitted by the treatment end of a fiber and/or surrounding tissues before pyrolysis occurs (rather waiting for a flash of white light resulting from pyrolytic burning, as required by the Dornier patent), for example by monitoring wavelengths indicative of temperature at the treatment end;
placement of the detector at locations other than the proximal end of the fiber (rather than at the proximal end, as required by the Dornier patent), for example by placing the detector at a bend in the fiber to take advantage of natural leakage without having to invasively tap and/or splice the fiber; and
modulation of the output of the laser (rather than control of the laser, as required by the Dornier patent), by downstream deflection/absorption/or fiber movement.

The improvements also include the following features, which may be used with the Dornier system, but which also may be used separately:
cleaning of the fiber following detection of excess temperature to enable reuse of the fiber,
inclusion of an additional detector to sense fiber position relative to the working channel of a catheter or introducer to prevent damage resulting from the fiber being pulled back into the catheter or introducer, and/or to take advantage of the protective effect of a Teflon™ or similar coating within the working channel in order to minimize damage caused by an overheating or burning fiber.

With respect to cleaning, the invention may utilize manual cleaning means such as wiping or dipping, or automatic cleaning by flushing the tip of the fiber with a cleaning fluid such as saline, either after thermal runaway has been detected or continuously during a procedure; and with or without feedback as to whether the fiber has been adequately cleaned.

Still further, the invention may be applied to the method disclosed in U.S. Provisional Application Ser. No. 60/996,918, filed Dec. 11, 2007, in which the fiber is caused to emit laser light at ultraviolet wavelengths during introduction of the catheter or fiber into a vein or blood vessel, thereby preventing the vessel wall from closing down or undergoing a spasm, after which the UV source is switched to a conventional treatment radiation source and treatment proceeds in accordance with the principles described above.

The above listed improvements may be used separately or in any combination with each other or with any elements of the system disclosed in the Dornier patent. Additional thermal runaway prevention measures, which may also be used with those of the present invention, are disclosed in copending U.S. patent application Ser. Nos. 11/510,691 and 11/714,785, by the same inventor and incorporated herein by reference. While the methods and apparatus of the invention may be used with a variety of surgical laser procedures, it is especially useful for urological and varicose vein treatment applications where thermal runaway has been a particular problem.

2. Description of Related Art

The Dornier patent discloses a method of controlling a surgical laser instrument by detecting radiation from pyrolitic burning of biological tissue. When burning of tissue is detected, the emitted radiation is guided back through the laser delivery fiber to a detector situated outside the patient, at the “proximal” end of the fiber, and the output of the laser is controlled by comparing the detected radiation in a particular spectral region with a threshold.

A detailed discussion of the causes and effects of burn back, particularly in the context of varicose vein treatment such as the EVLT® procedure of Diomed, is included in copending U.S. patent application Ser. Nos. 11/714,785 and 12/003,249. The effects include pain, nerve damage, adjacent tissue damage, vein perforation and creation of toxic fumes, all of which can cause considerable discomfort and present a significant risk to the health of the patient undergoing the treatment. Similar problems can also occur in other surgical laser applications.

It is of course well known in general to control a laser by feedback. The Dornier patent applies this technology to the specific case of tissue pyrolysis detection, and thus is especially intended to detect the “glow” that results when pyrolysis occurs. Because of this specific focus, the arrangement disclosed in the Dornier patent has several limitations when applied to surgical laser applications other than the one for which is was intended:
a. the system of Dornier is not designed to detect radiation emitted prior to tissue pyrolysis, or radiation from sources other than burning tissues;
b. the system of Dornier requires that the detector be at the proximal end of the delivery fiber; and
c. the feedback is used solely to control the laser, with no provision for deflection, absorption, or fiber movement based on the feedback.
   As a result, the Dornier system has a number of disadvantages:
a. The first disadvantage is that, by the time that pyrolytic glow or black body radiation is detected, substantial burn back, vein char or perforation may already have occurred. This is because damage from overheating is not only the result of pyrolysis, but also the result of heating of the fiber tip due to carbonization or build-up of debris on the tip. It turns out that fused silica, the core of the fiber which transmits laser energy, begins to highly absorb infrared laser radiation once the temperature at the tip exceeds 1000 degrees Celsius, well below the detection threshold of the Dornier system (which requires a flash of white light indicative of actual pyrolysis rather than high fiber temperatures). The absorption of infrared radiation can raise the temperature to well in excess of 5000 degrees Celsius before a flash is detected and the laser shut down, by which time injury to the patient and the need for expensive replacement of the fiber will already have occurred.
b. The second disadvantage is that placement of the detector at the proximal end of the delivery fiber, as required by Dornier, is vulnerable to background radiation/noise and losses in the delivery fiber, as well as losses due to tissues that might obscure or block radiation from the treatment area.
c. The third disadvantage is that Dornier's requirements of laser control and detection at the proximal end cannot be implemented without substantial, relatively expensive, modification of standard laser equipment, as well as splicing or invasively tapping into the delivery fiber. In practice, the need to replace fibers adds significantly to the cost of treatment since fibers need to be replaced relatively frequently.

An addition problem with the Dornier system and other conventional surgical laser system and methods is that, during laser surgery, surgeons can accidentally pull a laser fiber back into a delivery catheter such as an endoscope's working channel or a vein introducer and cause extensive damage to the catheter, or injury to the surgeon or patient. In the case of an endoscope, the working channel is typically a Teflon tube surrounded by image and illumination bundles. Once laser energy perforates this working channel, fluid will leak around the image bundles and distort the view of the image. The cost of this repair is typically $6000 to $8000 dollars. This is an especially common problem in urological applications such as stone management, during which the surgeon may accidentally pulls the fiber tip into the scope's working channel while lasing to breakup kidney stones. One endoscope company, ACMI, utilizes a camera feed back to detect a colored fiber buffer, but the feedback is used to control the laser rather than to prevent the fiber tip from being pulled back into the working channel, with the result that the laser frequently shuts off unintentionally due to poor visibility, extending the length of the surgical procedure and therefore both the cost and risk of surgery.

### SUMMARY OF THE INVENTION

It is accordingly a first objective of the invention to provide improved methods and apparatus for minimizing damage caused by overheating at the distal end of a therapeutic or surgical energy delivery device.

It is a second objective of the invention to provide a method and apparatus for rapidly and reliably detecting any overheating at a treatment site, and in particular a detection method and apparatus that minimizes background radiation/noise and losses in the delivery fiber.

It is a third objective of the invention to provide a method and apparatus for minimizing damage due to overheating at the treatment site, that does not necessitate modification to the laser and/or splicing or tapping into the delivery fiber.

It is a fourth objective of the invention to provide a method and apparatus which provides for cleaning and re-use of the fibers.

It is a fifth objective of the invention to provide a method and apparatus of the invention that prevents a fiber tip from accidentally being withdrawn into a catheter or introducer during lasing.

These objectives are achieved, in accordance with the principles of a preferred embodiment of the invention, by a laser delivery method and apparatus which monitors a temperature of a treatment area and provides a controller with a signal indicative of the temperature before burn back or thermal runaway occurs. The method and apparatus may monitor infrared or visible radiation that propagates in the delivery fiber, or may measure the temperature more directly at the distal end of the fiber. When a controller detects a high temperature, the output of the laser is modulated by absorption, deflection, or attenuation of the laser output, and/or a control signal is sent from the controller to the laser, for example, via a foot switch, door switch, safety interlock, laser internal microprocessor etc., to interrupt laser transmission. The interruption can be made permanent or momentary depending on the degree of damage to the catheter, introducer, vein or an endoscopes working channel.

The objectives of the invention are further achieved by placing the thermal runaway or burn back detector away from the proximal end of the fiber, and preferably near the treatment area so as to minimize background radiation/noise from, for example, bright operating room lights.

Whether the detector is situated at the proximal or distal end of the fiber, or in between, an especially advantageous embodiment of the invention is to place a photo detector near a bend in the delivery fiber to detect higher order propagation modes leaking from the bend, without the need to invasively tap or splice the fiber.

According to another preferred embodiment of the invention, a controller is situated between the delivery fiber and the laser source, and the controller includes devices for absorbing, deflecting, or otherwise attenuating the output of the laser in response to feedback from a detector, thereby eliminating the need to modify the laser to respond to the feedback.

The invention further provides for cleaning and re-use of fibers upon detection of overheating, before the fibers are destroyed by thermal runaway. The cleaning may be carried out manually upon activation of an alarm triggered by the temperature detector and removal of the fiber from the patient, or in vivo by manual or automatic dispensing of saline or other fluid can be applied to the treatment site. The fluid can also act to cool the tip during lasing treatment and cleaning. In cases where a continuous amount of saline can be applied, the fiber could be continuously cooled to prevent thermal runaway and still allow burn off of debris.

Finally, the invention provides for fiber position feedback. This may be accomplished from an endoscope by using its CCD camera and image detection software, or by encoding the fiber buffer by optically, mechanically, or electrically readable markings or other means. If the fiber is encoded, the output of a reader positioned on an introducer or catheter could be used to drive a relay or control signal to control or modulate the laser output, or to provide a warning of an approaching limit, whenever the fiber is withdrawn into the introducer or catheter. On the other hand, those skilled in the art will appreciate that if the working channel of the catheter or introducer is coated with Teflon™ or a similar protective material, it may actually be preferable to withdraw an overheating fiber into the working channel, where damage will be minimal due to the protective effect of the coating.

### BRIEF DESCRIPTION OF THE DRAWINGS

FIGS. 1-3 are schematic diagrams showing apparatus for minimizing damage caused by overheating or burn back at a therapeutic laser treatment site in accordance with the principles of various preferred embodiments of the present invention.

FIGS. 4 and 5 are respective graphs illustrating principles of temperature detection used by the apparatus and method of preferred embodiments of the present invention.

FIG. 6 is a schematic diagram illustrating a detector for radiation leaking from a bend in an optical fiber, according to the principles of a preferred embodiment of the present invention.

### DETAILED DESCRIPTION OF THE PREFERRED EMBODIMENTS

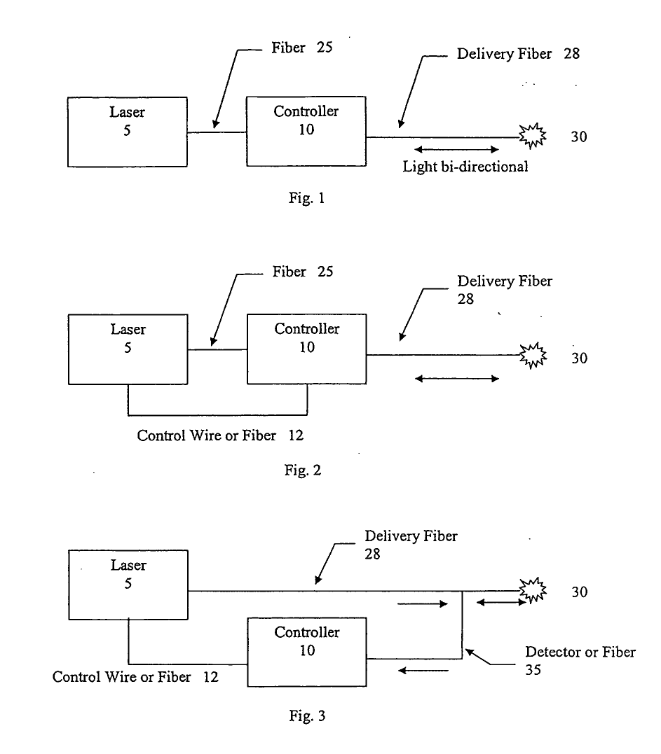

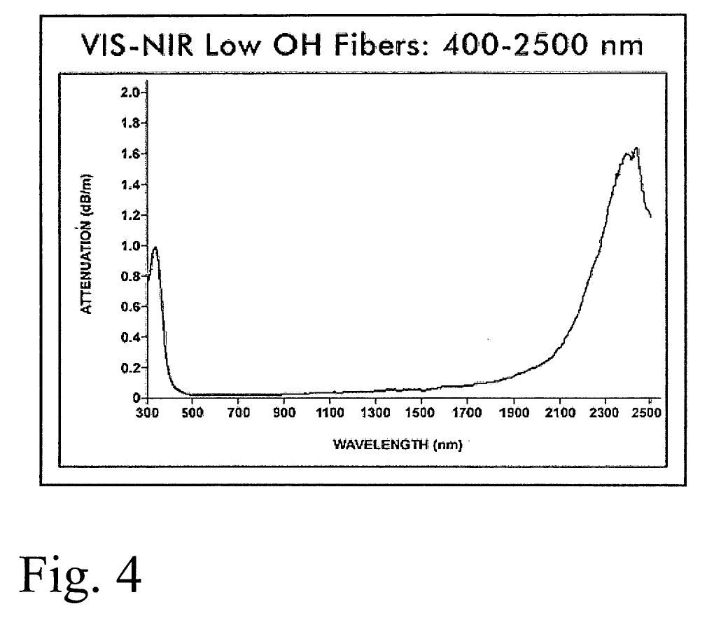

FIGS. 1-3 show feedback arrangements arranged to monitor temperatures in a treatment area at a distal end of a laser delivery fiber 28. In the embodiments of FIGS. 1 and 2, a controller 10 monitors temperature by detecting light at a proximal end of the delivery fiber 28, while in the embodiment of FIG. 3, temperature is monitored by detecting light at the distal end of the delivery fiber 28. In either case the temperature feedback is used by controller 10 to control the output of a laser 5, either by deflecting, absorbing, attenuating or otherwise modulating the output of the laser 5 as in FIG. 1, by combining output modulation with direct control of the laser apparatus as in FIG. 2, or by simply controlling the laser apparatus as in FIG. 3. In each case, the controller 10 may monitor wavelengths other those in the visible spectral region between 0.3 and 0.9 μm (“white light”), and in particular may monitor infrared radiation, which is indicative of temperature. The source of the radiation may be tissues or the fiber itself, which increases in temperature due to carbon build-up, and the controller 10 may take into account frequency or wavelength rather than just intensity in order to more accurately detect heating before thermal runaway or burn back occurs, rather than just the flash caused by burn back as in the Dornier patent discussed above.

The principles behind monitoring of temperature based on wavelength are well-known, although they have not be applied to detection of thermal runaway in the context of a laser delivery apparatus. In particular, there is an inverse relationship between wavelength and temperature, and therefor monitoring of the wavelength of radiation emitted by any heated body can be used as an indirect way to monitor temperature. The radiation emitted by the heated body is known as “black body radiation.” As explained below, the transmission capabilities of a typical delivery fiber are well within the range needed to detect increases in temperature indicative of thermal runaway.

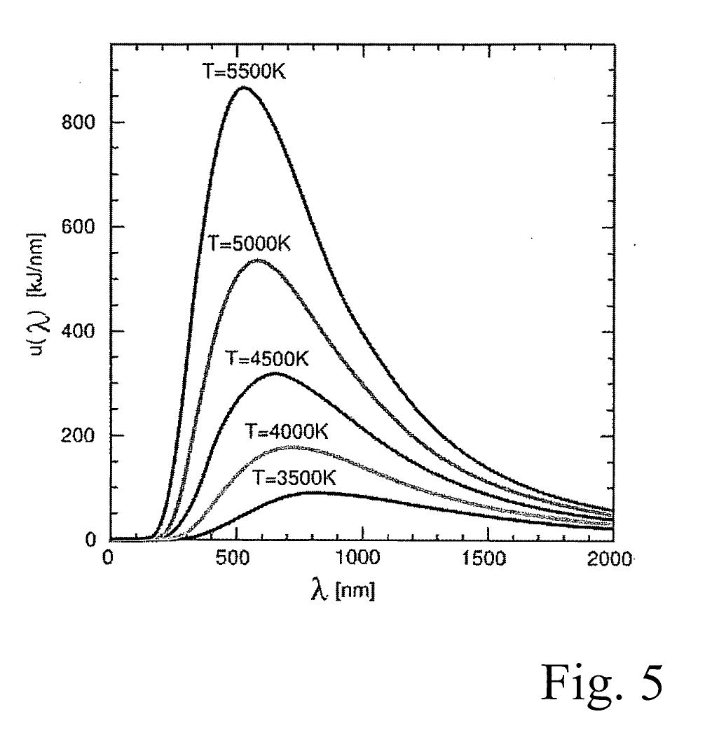

The optical detection range of the preferred apparatus and method will typically be limited by optical transmission of the fiber. For a silica fiber, the detection range is typically about 400-2100 nm. It is well-known to physicians that, when thermal runaway occurs, a bright white light can be seen emitting from the tip. From Black Body radiation curves illustrated in FIG. 4 and Wien's displacement law, the thermal runaway temperature is around 5500K or 5527 C, as illustrated in FIG. 5. Wien's displacement law is a law of physics that states that there is an inverse relationship between the wavelength of the peak of the emission of a black body and its temperature:

$${λ}_{max} = \frac{b}{T}$$

where λₘₐₓ is the peak wavelength in meters, T is the temperature of the blackbody in kelvins (K), and b is a constant of 2.897×10⁻³ m K. This relationship can be used to monitor the temperature by determining the wavelength of the peak of the detected black body radiation.

In the feedback arrangement of FIG. 1, controller 10 is connected to the laser 5 by a jumper fiber 25. In this configuration, light from the distal end of the fiber is transmitted back through the delivery fiber 28, or through a catheter, introducer, or additional fiber, and is detected by controller 10. Instead of controlling the laser as required by the Dornier patent, controller 10 deflects, absorbs, or attenuates laser energy coming from fiber 25 when an over temperature is detected. The deflected energy may, for example, be safely directed into a heat sink. This arrangement has the advantage that it is not necessary to modify the laser in order to achieve control of the delivered laser energy in response to temperature feedback.

FIG. 2 shows a variation of the feedback arrangement of FIG. 1 in which, in addition to or instead of deflecting, absorbing, attenuating, or otherwise modulating laser energy from fiber 25, the controller sends an interrupt signal to the laser via control wire or fiber 12 if the temperature is too high. The signal may be used to disable the laser foot switch, door interlock, or the enable signal from the laser's 5 internal microprocessor in order to immediately terminate the delivery of laser energy upon detection of imminent or actual thermal runaway.

In this embodiment, the interrupt signal can be based on the intensity or level of light produced by thermal runaway as well as duration of emission. At some determined value of light level and/or duration, the detector circuit can send an alarm signal indicating a dirty fiber or thermal runaway. Additionally, the alarm signal could be used to place the laser in standby by interrupting door interlock. Preferably, the operator would be allowed to adjust level or duration parameters for their particular application.

FIG. 3 shows a variation of the arrangements of FIGS. 1 and 2, in which the need for jumper fiber 25 is eliminated and an external sense fiber, photo sensor, or other temperature detector 35 is placed away from the proximal end of the delivery fiber 28, rather than at the proximal end as required by the Dornier patent. This variation is especially advantageous in that it eliminates interference caused by external lighting, and/or noise or losses resulting from transmission back through the delivery fiber. Since the feedback signal is cleaner, there is a lower possibility of false indications and a lower threshold can be used to indicate thermal runaway or trigger an alarm/shut down of the laser.

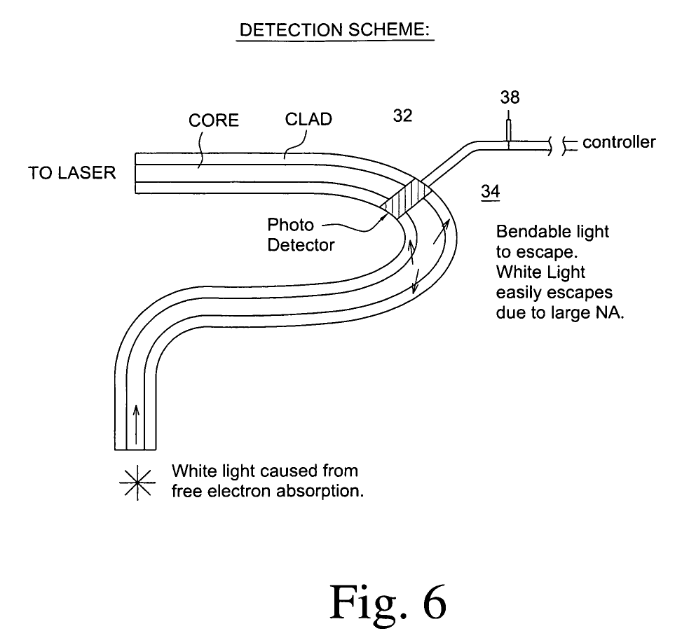

Each of the embodiments illustrated in FIGS. 1-3 depends on detection of radiation in the fiber core. This can be done by traditional tapping methods such as a fusion splice, but requires modification of the fiber resulting in increased manufacturing cost and loss of strength of the fiber. However, according to an especially advantageous embodiment of the invention, the property that light emitted from the distal end of the fiber creates a lot of weakly guided high order propagation modes is utilized to eliminate the need for splicing or other modifications of the fiber. In particular, a slight bend will readily leak out the thermal runaway light with minimal loss from the primary laser source and therefore, as illustrated in FIG. 6, the light can be detected simply by placing a photo detector 32 and the bend 34 in the fiber and connecting the photo detector 32 to controller 10 by a wire or fiber 38. The advantage is that the fiber does not have to be altered in any way and detection is non-contact.

An additional advantage of the feedback arrangements illustrated in FIGS. 1-3 is that the delivery fiber can be safely moved back to create a larger heat zone, which lowers the average power density and permits the laser output to be increased, thereby treating a larger area and/or increasing the speed of treatment.

It will be appreciated that the overheating detection/feedback apparatus of FIGS. 1-3 can be used with or without an introducer. In the arrangement of FIGS. 1 and 2, if an introducer is used, the introducer may act as a waveguide for radiation generated by burning tissues. In either case, the clarity of fluid in the introducer may be detected to check for proper flushing or to indirectly detect effects of overheating, and electrical feedback may be included as an alternative to, or in addition, to optical feedback. The electrical feedback may take the form of a thermo-couple or other heat-sensitive electrical device, or a photosensor for detecting visible radiation resulting from overheating or burn back.

In addition to providing control or modulation of the laser output, the invention provides for cleaning of the fiber, either manually or automatically, upon activation of an alarm in response to a temperature increase in the treatment area.

In the case of manual cleaning, the surgeon or operator of the laser should clean the fiber of debris and visually test the fiber by lasing to insure no more thermal runaway is present before resuming a procedure after detecting an increase in lasing temperature or thermal runaway. Cleaning by mechanical means such as wiping with alcohol may not be sufficient to remove all the debris, and therefore a preferred method is to dip the tip into a fluid such as saline and fire the laser for a few seconds to clean the tip and then raise the fiber out of the fluid and test in air. If the fiber shows no sign of thermal runaway, then the surgical procedure or treatment may be resumed. If not further cleaning in the fluid may be carried out. Use of a fluid is a simple way to prevent the fiber tip from creating thermal runaway, but at the same time allows the tip to get hot enough to burn off debris. To immediately resume treatment, the fluid, such as saline, should be sterile. Although a fluid is preferred, other cooling means such as air could be used to lower the temperature during cleaning.

In cases where the detector is already arranged to detect thermal runaway at some predetermined intensity threshold, as described for example in the Dornier patent, a second lower threshold could bet set to where the operator is automatically notified when the fiber is clean enough of debris.

On the other hand, in some cases, it may not be practical to remove the fiber from the surgical site. Instead, it may be desirable to clean fiber in vivo. Where saline or other fluid can be applied to the treatment site, the fluid can also act cool the tip during lasing treatment and cleaning. The fluid could be manually dispensed via a syringe or a continuous fluid drip to the treatment site. Alternatively, the detector circuit could continuously detect a thermal runaway and signal an automatic fluid dispenser. The dispenser would dispense just enough fluid to stop a thermal run away and at same time allow burn off of debris. A fiber with a coaxial tube to pass fluid would be a preferred means to deliver cooling directly to the fiber tip.

Finally, with respect to embodiment involving cooling, in procedures where a continuous amount of saline may be applied, the fiber could be continuously cooled, for example through a coaxial tube, thereby preventing thermal runaway while still allowing burn off of debris. However, the continuous cooling may have adverse effects in thermal laser treatments.

A final feature of the invention is to provide for fiber position feedback in addition to or instead of temperature feedback. One way to accomplish fiber position feedback is to utilize the CCD camera of an endoscope and corresponding image detection software, although difficult issues surrounding variability image views would have to be addressed. For example, if a camera and software are being used to detect a fiber's colored buffer or its laser emissions, differences in buffer color, laser intensity, laser wavelength, field of view, clarity, background illumination intensity, etc., would need to be accounted for.

Alternatively, the fiber buffer could be encoded by optical, mechanical, or electrical means, and an appropriately positioned reader used to detect the encoding and therefore the fiber position. For example, magnetic barcode could be affixed to the fiber buffer to identify the position of the fiber inside an endoscope. The reader preferably could be attached to the fiber holder or made a part thereof. Typically, fiber holders are secured to the entrance of the working channel via a luer fitting common to most endoscopes or catheters.

In this embodiment of the invention, the output of the reader could be used to drive a relay or control signal to control the laser, for example by providing a signal to a laser door or footswitch interlock. Also, the encoded fiber could be placed into the scope's working channel and read to determine the proximal and distal positions. Once the positions are determined, the read signal could provide an advanced warning (beeps) that a limit is approaching, or to remotely disable the laser when the surgeon withdraws it into the working channel of an endoscope in order to reposition the scope. On the other hand, since the working channel of a catheter or introducer is typically coated with a protective material such as Teflon™, it may be advantageous in some applications to utilize the protective properties of the material to minimize damage due to overheating or burning by withdrawing the fiber into the working channel where damage will be minimal.

Another use for a fiber position sensor would be to eliminate the need for pre-mapping before a surgical procedure. In vascular work, such as EVLT, laser dosage must change as the vein diameter increases. As such, the surgeon must pre-map the patient leg using ultrasound and determine the dosage at various points along the vein. This takes considerable time and effort for the surgeon. Since the position sensor can be used to determine its relative position in the vein, its output signal could be coupled with the ultrasound signal to control laser delivery without pre-mapping. Also, at the same time the rate of withdraw speed of the fiber inside the vein could also be detected and further used to control laser dosage.

Each of the methods and/or devices described above may be applied to a modified treatment method in which ultraviolet radiation is applied to the treatment area before conventional treatment radiation is applied. As explained in U.S. Provisional Application Ser. No. 60/996,918, filed Dec. 11, 2007, in all of the existing treatment methods, a problem arises in that, when pressure is exerted on the inner vessel wall upon introduction of a catheter or even a bare fiber, the vessel wall may close down or spasm. If the vessel closes down, the procedure cannot be performed for that day. To solve this problem, the invention proposes using a UV source to aid in introducing the catheter or fiber into the vein or blood vessel. The UV source causes dilation of the vessel, thereby preventing it from closing down before the treatment procedure can be performed. The UV radiation is applied by causing the fiber to emit laser light at ultraviolet wavelengths during introduction of the catheter or fiber into the vessel, and then when the catheter and/or fiber is in place, the UV source (which may be a laser, diode, mercury lamp etc.) is switched to a conventional treatment radiation source, such as a visible laser light source so that treatment can commence before the vessel has a chance to collapse. The use of UV radiation for vasodilation has previously been proposed in U.S. Patent Publication No. 2004/009213, but for the purpose of stretching an occluding thrombus rather to keep a vessel open during positioning of a catheter or fiber.

Having thus described various preferred embodiments of the invention in sufficient detail to enable those skilled in the art to make and use the invention, it will nevertheless be appreciated that numerous variations and modifications of the illustrated embodiment may be made without departing from the spirit of the invention, and it is intended that the invention not be limited by the above description or accompanying drawings, but that it be defined solely in accordance with the appended claims.

What is claimed is:

1. A method for carrying out surgical procedures using a laser, comprising the steps of connecting an optical fiber delivery system to the laser and positioning an output end of the optical fiber at the treatment site, providing a detector disposed to monitor radiation emitted when overheating of the fiber or tissues at the treatment site has occurred or is about to occur, operating the laser to perform surgery by supplying treatment radiation to the surgical site through the output end of the optical fiber, providing an indication that overheating has occurred or is about to occur based on a signal output by the detector, and shutting down or modulating an output of the laser before burn back occurs based on the overheating indication.

2. A method as claimed in claim 1, wherein said step of controlling an output of said laser includes the step of modulating said output of said laser by absorbing, deflecting, or attenuating said laser light in response to detection of said temperature.

3. A method as claimed in claim 1, wherein said step of controlling an output of said laser includes the step of modulating said output of said laser in response to a detected wavelength of radiation emitted at said treatment site, said wavelength being indicative of said temperature.

4. A method as claimed in claim 1, further comprising the step of generating an alarm signal upon detection of a temperature indicative of overheating.

5. A method as claimed in claim 1, further comprising the step of applying UV radiation to the treatment site before operating the laser to supply said treatment radiation to the treatment site.

6. A method as claimed in claim 1, wherein the optical fiber is positioned at the treatment site through an introducer or catheter.

7. A method as claimed in claim 1, wherein the optical fiber is positioned at the treatment site without an introducer or catheter.

8. A method for carrying out surgical procedures using a laser, comprising the steps of connecting an optical fiber delivery system to the laser and positioning an output end of the optical fiber at the surgical site, supplying UV radiation to the treatment site through the output end of the optical fiber, and thereafter operating the laser to perform surgery by supplying treatment radiation to the surgical site through the output end of the optical fiber.

9. A method as claimed in claim 1, wherein the optical fiber is positioned at the treatment site through an introducer or catheter.

10. A method as claimed in claim 1, wherein the optical fiber is positioned at the treatment site without an introducer or catheter.
